(19) United States
(12) Patent Application Publication
(10) Pub. No.: US 2020/0111558 A1
(43) Pub. Date: Apr. 9, 2020

(54) INFORMATION PROCESSING APPARATUS, MEDICAL IMAGE DISPLAY APPARATUS, AND STORAGE MEDIUM

(71) Applicant: Konica Minolta, Inc., Tokyo (JP)

(72) Inventors: Hiroaki MATSUMOTO, Yokohama-shi (JP); Shinsuke KATSUHARA, Tokyo (JP); Hitoshi FUTAMURA, Tokyo (JP); Satoshi KASAI, Tokyo (JP)

(21) Appl. No.: 16/577,755

(22) Filed: Sep. 20, 2019

(30) Foreign Application Priority Data
Oct. 5, 2018 (JP) 2018-190165

(51) Int. Cl. G16H 30/20; G16H 50/20; G06T 7/00; G16H 10/60

(52) U.S. Cl. CPC G16H 30/20; G16H 50/20; G16H 30/40; G16H 10/60; G06T 7/0012

(57) ABSTRACT

An information processing apparatus includes a hardware processor. The hardware processor obtains an abnormal shadow candidate detection result generated based on a medical image obtained by a medical image generation apparatus. Based on the obtained abnormal shadow candidate detection result, the hardware processor determines the number of image interpreters who interpret the medical image. The hardware processor outputs the determined number of image interpreters.

### BACKGROUND

### 1. Technological Field

The present disclosure relates to an information processing apparatus, a medical image display apparatus, and a storage medium.

### 2. Description of the Related Art

There is known in the medical field a computer aided diagnosis (CAD) system that automatically detects abnormal shadow candidates in medical images, and outputs the medical images with the visibility of the detected abnormal shadow candidates increased.

Image interpreters, such as doctors, interpret the images including the abnormal shadow candidates detected by the CAD, and make final decisions on whether or not the abnormal shadow candidates in the images are abnormal shadows indicating lesions, such as tumors and calcification.

With the aim of improving overall working efficiency and accuracy in image interpretation, there is provided a method for determining doctors who are in charge of interpreting medical images on the basis of analysis results of the medical images by the CAD and information on doctors' qualifications. (For example, see JP 2009-157527 A.)

The level of difficulty in image interpretation varies from medical image to medical image. Hence, in order to improve accuracy in image interpretation, it is preferable in some cases that a plurality of image interpreters interpret one image. However, overall working efficiency will not be high if every medical image including, for example, a medical image that is taken in a medical examination and does not include lesions is interpreted by a plurality of image interpreters.

### SUMMARY

Objects of the present disclosure include improving accuracy in image interpretation in interpreting medical images using the CAD without decreasing overall working efficiency.

In order to achieve at least one of the abovementioned objects, according to a first aspect of the present invention, there is provided an information processing apparatus including a hardware processor that: obtains an abnormal shadow candidate detection result generated based on a medical image obtained by a medical image generation apparatus; based on the obtained abnormal shadow candidate detection result, determines the number of image interpreters who interpret the medical image; and outputs the determined number of image interpreters.

According to a second aspect of the present invention, there is provided a medical image display apparatus that connects to the information processing apparatus and a medical image generation apparatus and displays the medical image, the medical image display apparatus comprising a hardware processor that causes a display to display a list screen of a case in which the medical image is associated with one or more image interpreters based on the image interpreter assignment information.

According to a third aspect of the present invention, there is provided a non-transitory computer-readable storage medium storing a program to cause a computer to: obtain an abnormal shadow candidate detection result generated based on a medical image obtained by a medical image generation apparatus; based on the obtained abnormal shadow candidate detection result, determine the number of image interpreters who interpret the medical image; and output the determined number of image interpreters.

### BRIEF DESCRIPTION OF THE DRAWINGS

The advantages and features provided by one or more embodiments of the present invention will become more fully understood from the detailed description given hereinbelow and the appended drawings which are given by way of illustration only, and thus are not intended as a definition of the limits of the present invention, wherein:

FIG. 1 shows an example of system configuration of a medical image display system;

FIG. 2 shows an example of functional configuration of an information processing apparatus;

FIG. 3 shows an example of functional configuration of a medical image display apparatus;

FIG. 4 is a diagram to explain FCN (Fully Convolutional Networks);

FIG. 5 is a flowchart showing a series of processes that are performed by the information processing apparatus;

FIG. 6 is a flowchart showing a process of determining the number of image interpreters in FIG. 5;

FIG. 7A is an example of a candidate doctor table that is used in a process of assigning medical images to image interpreters;

FIG. 7B is an example of a priority order table that is used in the process of assigning medical images to image interpreters;

FIG. 8 is a flowchart showing a process of displaying medical images that is performed by the medical image display apparatus;

FIG. 9 shows an example of a list screen;

FIG. 10 is a flowchart showing the process of determining the number of image interpreters according to a first modification;

FIG. 11 is a flowchart showing another example of the process of determining the number of image interpreters according to the first modification;

FIG. 12 is a flowchart showing the process of determining the number of image interpreters according to a second modification;

FIG. 13 is a flowchart showing the process of determining the number of image interpreters according to a third modification;

FIG. 14 is a flowchart showing the process of determining the number of image interpreters according to a forth modification;

FIG. 15 is a flowchart showing the process of determining the number of image interpreters according to a fifth modification;

FIG. 16 is a flowchart showing another example of the process of determining the number of image interpreters according to the fifth modification;

FIG. 17 is a flowchart showing another example of the process of determining the number of image interpreters according to the fifth modification; and

FIG. 18 shows an example of the list screen according to a sixth modification.

### DETAILED DESCRIPTION OF EMBODIMENTS

Hereinafter, one or more embodiments of the present invention will be described with reference to the drawings. However, the scope of the present invention is not limited to the disclosed embodiments.

### [Configuration of Medical Image Display System 100]

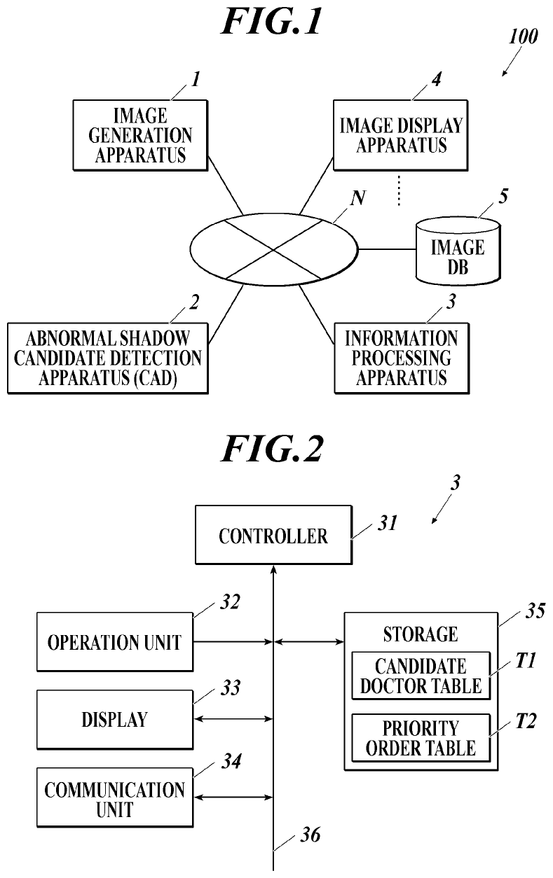

FIG. 1 shows system configuration of a medical image display system 100 according to this embodiment.

The medical image display system 100 is a system in which: a medical image is taken; on the basis of the medical image, abnormal shadow candidate(s) is detected; and the detection result along with the medical image is provided to an image interpreter(s).

As shown in FIG. 1, the medical image display system 100 includes an image generation apparatus 1, an abnormal shadow candidate detection apparatus (CAD) 2, an information processing apparatus 3, an image display apparatus 4, and an image database (DB) 5. These apparatuses/components 1 to 5 connect to each other for data exchange through a communication network N, such as a local area network (LAN), built in a medical facility. The communication network N conforms to Digital Imaging and Communication in Medicine (DICOM) standard. The number of apparatuses/components 1 to 5 is not limited. For example, the CAD 2, the information processing apparatus 3, and the image DB 5 can be included in one computer.

Hereinafter, configuration of each of the apparatuses/components 1 to 5 is described.

### [Image Generation Apparatus 1]

The image generation apparatus 1 is a medical image generation apparatus that takes images of human bodies (examinees) and generates digital data of the taken images (medical images). As the image generation apparatus 1, modalities, such as an X-ray radiography apparatus using a CR (Computed Radiography), an X-ray radiography apparatus using an FPD (Flat Panel Detector), a CT (Computed Tomography) apparatus, an MRI (Magnetic Resonance Imaging) apparatus, a cassette-type image reading apparatus, and a film digitizer, can be used. An example of the image generation apparatus 1 is an X-ray radiography apparatus. The X-ray radiography apparatus generates data of X-ray images, such as a chest X-ray image and an abdominal X-ray image.

The image generation apparatus 1 conforms to the DICOM standard. The image generation apparatus 1 can accept inputs of various kinds of information, such as patient information and examination information, to be attached to a generated medical image from outside. The image generation apparatus 1 can also automatically generate the information. The patient information includes patient identification information (e.g. patient ID) for identifying a patient, patient name, sex, and date of birth. The examination information includes examination identification information (e.g. examination ID) for identifying an examination, date of examination, examination condition (examined body part, body position, and an imaging direction, e.g. from the front or the side of the body), and a type of the modality.

The image generation apparatus 1 attaches, as header information, the patient information, the examination information, a unique ID (UID) for identifying the medical image, and the like to the generated medical image, sends the medical image with the header information to the image DB 5 through the communication network N, and stores and accumulates the same in the image DB 5. The image generation apparatus 1 can send the medical image directly to the CAD 2 and the information processing apparatus 3. In a case where an apparatus that does not conform to the DICOM standard is used as the image generation apparatus 1, a DICOM conversion device (not illustrated) can be used to input the information to be attached to the medical image to the image generation apparatus 1.

### [Abnormal Shadow Candidate Detection Apparatus (CAD) 2]

The CAD 2 is a computer that analyzes the medical image provided by the image generation apparatus 1, thereby performing a process of detecting abnormal shadow candidates. The CAD 2 includes: a central processing unit (CPU); a random access memory (RAM); a storage, such as a hard disk drive (HDD); and a communication unit, such as a LAN card.

The storage of the CAD 2 stores detection programs using a detection algorithm and corresponding to types of abnormal shadows (lesions). The CPU of the CAD 2 detects, in cooperation with the detection programs stored in the storage, an abnormal shadow candidate(s) in the medical image input through the communication unit. The CPU of the CAD 2 detects, for example, abnormal shadow candidates of nodular shadows and cardiac hypertrophy in chest X-ray images.

As the detection algorithm for detecting abnormal shadow candidates, an algorithm known to the public can be adopted. For example, fully convolutional networks (FCN), which is a deep learning model, can be used. Details of an FCN is described later.

After finishing the process of detecting abnormal shadow candidates with the detection algorithm, the CAD 2 generates an abnormal shadow candidate detection result (hereinafter called CAD information). The CAD information includes: positional information of the region (contour) of each detected abnormal shadow candidate; the type of each detected abnormal shadow candidate (e.g. nodular shadows and cardiac hypertrophy); the number of the detected abnormal shadow candidates; the level of severity of each abnormal shadow candidate as a disease (e.g. the risk of death); and an abnormal shadow probability indicating a probability that the abnormal shadow candidate is an abnormal shadow. The CAD 2 attaches the generated CAD information to the header information of the medical image on which the process of detecting abnormal shadow candidates has been performed, and sends the medical image to the image DB 5 and/or the information processing apparatus 3 through the communication unit. The CAD information may be accumulated in the image DB 5 as a file separate from the corresponding medical image and associated with the corresponding medical image such that they are recognized to correspond to each other.

### [Information Processing Apparatus 3]

The information processing apparatus 3 is an apparatus that determines, on the basis of the medical image generated by the image generation apparatus 1 and the CAD information of the medical image analyzed by the CAD 2, the number of image interpreters who interpret the medical image.

As shown in FIG. 2, the information processing apparatus 3 includes a controller 31 (hardware processor), an operation unit 32, a display 33, a communication unit 34, and a storage 35 that connect to each other through a bus 36.

The controller 31 includes a CPU and a RAM. The CPU of the controller 31 reads out various programs stored in the storage 35, such as a system program and a process program, loads them into the RAM, and performs various processes in accordance with the loaded programs.

The controller 31 performs, for example, a process of determining the number of image interpreters, a process of assigning medical images to image interpreters, and a process of outputting image interpreter assignment information.

The operation unit 32 includes: a keyboard equipped with character keys, numeric keys, and various function keys; and a pointing device, such as a mouse. The operation unit 32 outputs, to the controller 31, press signals of pressed keys of the keyboard and operation signals by the mouse being operated as input signals.

The display 33 includes a monitor, such as a cathode ray tube (CRT) or a liquid crystal display (LCD), and displays various screens in accordance with instructions of display signals input by the controller 31.

The communication unit 34 includes a LAN card, and exchanges data with external apparatuses connecting to the communication network N through a switching hub.

The storage 35 includes a hard disk drive (HDD) and/or a nonvolatile semiconductor memory. The storage 35 stores various programs, data, and the like as described above.

For example, the storage 35 stores a candidate doctor table T1 and a priority order table T2 both of which are used in a process of assigning medical images to image interpreters described below.

### [Image Display Apparatus 4]

The image display apparatus 4 is a medical image display apparatus that obtains a medical image specified by operations of an image interpreter and the corresponding CAD information from the image DB 5, and displays the same. The medical image display apparatus is sometimes used to display a medical image for explaining to a patient, as well as for interpreting the medical image. When an image interpreter displays a medical image for explaining to his/her patient, the medical image display apparatus can be switched to a mode in which the CAD information is not displayed and only the medical image and information confirmed by the doctor are displayed. The image interpreter thus can explain to the patient without making the patient anxious unnecessarily.

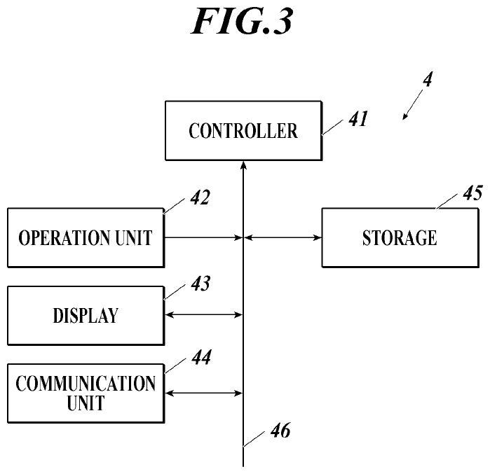

FIG. 3 shows an example of functional configuration of the image display apparatus 4.

As shown in FIG. 3, the image display apparatus 4 includes a controller 41 (hardware processor), an operation unit 42, a display 43, a communication unit 44, and a storage 45 that connect to each other through a bus 46.

The controller 41 performs, for example, a process of displaying medical images, which is described later, and displays various screens on a monitor, such as a CRT or an LCD.

### [Image DB 5]

The image DB 5 accumulates medical images generated by the image generation apparatus 1 and medical images with CAD information attached thereto.

CAD information may be accumulated in the image DB 5 as a file separate from the corresponding medical image and associated with the corresponding medical image such that they are recognized to correspond to each other. This is performed as follows. The image DB 5 has an image management table that stores management information on each medical image stored in the image DB 5. The information on each medical image is stored as one record in the image management table. The management information includes UID, patient information, examination information, and file information (e.g. file names, file locations, date of updates, and file sizes, etc. of the medical image and the corresponding CAD information).

When the medical image is received from the image generation apparatus 1, the received medical image is stored in the image DB 5. On the basis of the header information of the received medical image, the management information is generated and stored in the image management table. When the CAD information is received from the CAD 2, the received CAD information is stored in the image DB 5, and a record of which UID agrees with the CAD information in the image management table is retrieved, and the file name, the file location, and the like of the CAD information are written in the retrieved record. Thus, the medical image and the CAD information generated from the medical image are associated with each other, and stored in the image DB 5 so as to be retrievable.

### [Operation of the Medical Image Display System 100]

Next, the operation of the medical image display system 100 is described.

### [Process by the CAD 2]

As described above, the CAD 2 performs a process of detecting abnormal shadow candidates and generates the CAD information.

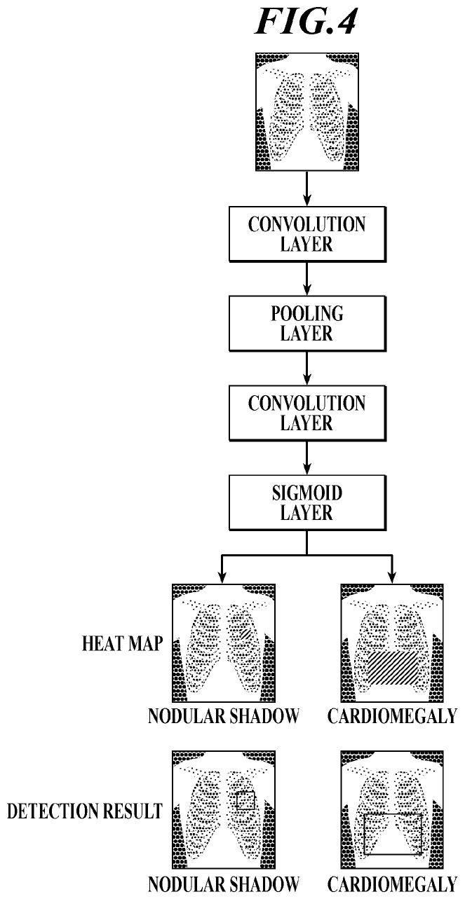

Here, FCN, which is an example of the detection algorithm performed by the CAD 2 to detect abnormal shadow candidates, is described. FIG. 4 shows an example of configuration of a classifier (network) using FCN. In a process using FCN, a large number of convolution layers that perform image filtering and a large number of pooling layers that sample outputs of the convolution layers are repeated, and a heat map of the input medical image is output. The heat map has values that range from 0 to 1 for respective regions of an input image and indicate probabilities of the regions being a lesion (abnormal shadow). Parameters for the convolution layers are optimized beforehand through a learning process using learning data labeled types and regions of lesions.

By performing the process using FCN, a heat map is output for each type of lesion to be diagnosed. The heat map shows probabilities indicating respective points on an image being a lesion.

A lesion is detected if a probability value in the heat map exceeds a predetermined threshold. The abnormal shadow probability is determined on the basis of a predetermined threshold and the probability value in the heat map. For example, if “V<(1.0+T)±2.0” holds, wherein V represents a probability value in the heat map, and T represents a threshold value being 0.5, it is determined that determination of whether or not the region is an abnormal shadow is difficult to be made. A triage level that indicates the level of urgency of the image interpretation is determined for each type of lesion. For example, the triage level for nodular shadows is set high, because the nodular shadows indicate a possibility of lung cancer and requires early diagnosis and treatment.

### [Processes by the Information Processing Apparatus 3]

The information processing apparatus 3 determines the number of image interpreters who are in charge of interpreting the medical image on the basis of the medical image and the CAD information of the medical image. The information processing apparatus 3 assigns an image interpreter(s) of the determined number to interpret the medical image, and outputs the assignment result (image interpreter assignment information) to the medical image display apparatus 4. This leads to improvement of efficiency in image interpretation.

FIG. 5 is a flowchart showing a series of processes that are performed by the information processing apparatus 3.

The controller 31 performs the processes in FIG. 5 in cooperation with the programs stored in the storage 35.

### <Process of Determining the Number of Image Interpreters>

First, the controller 31 performs the process of determining the number of image interpreters (Step S11) to determine the number of doctors (image interpreters) who interpret a medical image as an interpretation target.

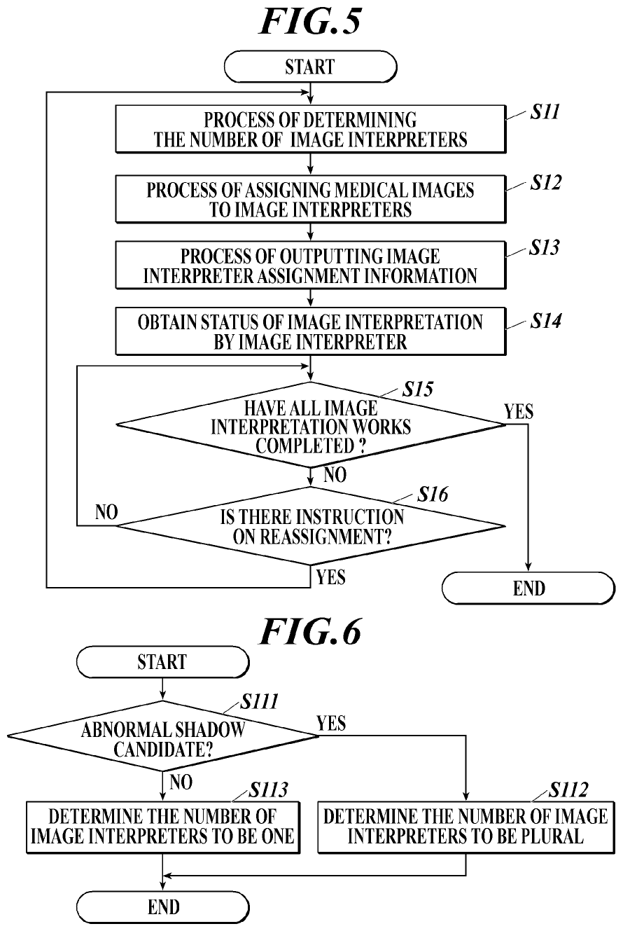

FIG. 6 shows a flowchart of the process of determining the number of image interpreters.

The controller 31 obtains the CAD information of the medical image as the interpretation target, and determines, as shown in FIG. 6, whether or not an abnormal shadow candidate is present according to the obtained CAD information (Step S111). If an abnormal shadow candidate is present (Step S111: YES), the controller 31 determines the number of image interpreters to be plural (Step S112). If an abnormal shadow candidate is absent (Step S111: NO), the controller 31 determines the number of image interpreters to be one (Step S113).

In the process of determining the number of image interpreters, the controller 31 determines the number of image interpreters according to whether or not an abnormal shadow candidate is present. Because only medical images that are highly likely to include an abnormal shadow(s) are interpreted by a plurality of image interpreters, accuracy in image interpretation is improved, and increase of time for image interpretation is restrained.

### <Process of Assigning Medical Images to Image Interpreters>

Referring back to FIG. 5, the controller 31 performs a process of assigning medical images to image interpreters (Step S12). In this process, the controller 31 determines an image interpreter(s) who will actually interpret the medical image, the image interpreter(s) of the number determined by the process of determining the number of image interpreters (i.e. assigns the image interpreter(s) to interpret the medical image), and generates image interpreter assignment information in which the medical image is associated with the image interpreter(s) of the determined number.

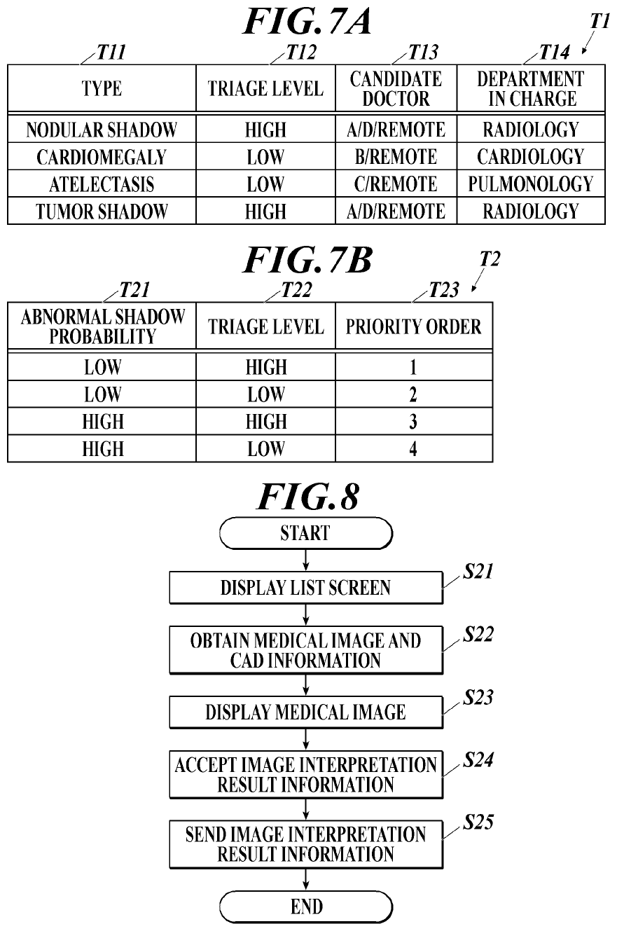

In the process of assigning medical images to image interpreters, the controller 31 obtains, by referring to a preset candidate doctor table T1 (FIG. 7A), the triage level for every detected type of abnormal shadow candidate.

FIG. 7A shows an example of the candidate doctor table T1.

As shown in FIG. 7A, the candidate doctor table T1 is a table that has items of: type T11; triage level T12; candidate doctor T13; and department in charge T14. In the candidate doctor table T1, each type of lesion is associated with the triage level, the candidate doctor(s), and the department in charge.

In the type T11, names of lesions (abnormal shadows) detected by the CAD 2 are shown.

In the triage level T12, the triage level is set to be “high” or “low” according to the lesion shown in the type T11.

In the candidate doctor T13, a name(s) of a doctor(s) who can be in charge of the lesion shown in the type T11 is shown. In the candidate doctor T13, as well as a doctor(s) who belongs to a medical facility where the medical image has been taken, a doctor(s) who belongs to any other medical facility or an image interpretation company that consists of image interpretation specialists may be shown. Herein, doctors who interpret medical images in locations remote from the medical facility where the medical images have been taken are shown as “remote” instead of the names of the doctors. For example, with respect to nodular shadows, a doctor A, a doctor B, and a doctor of remote image interpretation are set as doctors who can be in charge.

In the department in charge T14, a department(s) that is in charge of examining the lesion shown in the type T11 (a department to which the candidate doctor(s) shown in T13 belongs) is shown.

Next, the controller 31 determines, by referring to a preset priority order table T2 (FIG. 7B), a rank of each detected abnormal shadow candidate in the priority order according to the combination of the abnormal shadow probability and the triage level for the detected abnormal shadow candidate. Here, if a plurality of types of abnormal shadow candidates has been detected, the rank in the priority order is obtained for each type.

FIG. 7B is an example of the priority order table T2.

As shown in FIG. 7B, the priority order table T2 is a table that has items of the abnormal shadow probability T21, the triage level T22, and the priority order T23. In the priority order table T2, the priority order is set on the basis of the combination of the abnormal shadow probability and the triage level.

In the abnormal shadow probability T21 and the triage level T22, “high” or “low” is set. In the priority order T23, ranks from 1 to 4 are set according to the combination of the abnormal shadow probability and the triage level. Although the priority order is set according to the combination of the abnormal shadow probability and the triage level, the way of determining the priority order is not limited to this. For example, if there is information on a past examination of the patient of the medical image, a rank in the priority order may be determined on the basis of the information on the past examination. If there is a medical image of the patient taken in the past, the rank in the priority order may also be determined, on the basis of a comparison between the current image and the past image, in descending order of the change from the past.

Next, the controller 31 determines a doctor(s) who is in charge of interpreting each abnormal shadow candidate in descending order of the ranks in the priority order.

More specifically, with respect to the first abnormal shadow candidate, the controller 31 refers to the candidate doctor table T1 (FIG. 7A) and selects a doctor(s) who is in charge of the first abnormal shadow candidate among the candidate doctors set in T13. Here, the controller 31 selects one or plural doctors according to the determined number of doctors for the abnormal shadow candidate.

It is preferable that a doctor(s) who is most available among the selectable candidate doctors be selected prior to the other doctors. In consideration of each doctor's skill in image interpretation, each candidate doctor can be associated beforehand with a certain type(s) of abnormal shadow. For example, in selecting a doctor for an abnormal shadow candidate high in the priority order, it is preferable that a skilled or well-experienced doctor be selected prior to the other doctors. A difficult case may be assigned to a doctor who has a high percentage of accurate diagnoses on the basis of diagnostic accuracy of each doctor in the past. Furthermore, the schedule of the doctor who has a high percentage of accurate diagnoses can be kept open so that the doctor can be assigned difficult cases, without being assigned other ones, such as a case of which CAD information indicates a high abnormal shadow probability. As described above, the amount of works assigned to each doctor may be adjusted.

Next, with respect to the second abnormal shadow candidate, the controller 31 refers to the candidate doctor table T1 (FIG. 7A) and selects a doctor(s) to be in charge of the second abnormal shadow candidate among the candidate doctors set in T13. Here, the controller 31 selects one or plural doctors according to the determined number of doctors for the abnormal shadow candidate.

If the doctor who has been selected for the first abnormal shadow candidate can be also selectable for the second abnormal shadow candidate, it is preferable that the doctor be selected for the second abnormal shadow candidate prior to the other doctors.

As described above, the controller 31 selects a doctor(s) for every detected abnormal shadow candidate, and determines the selected doctor(s) to be the image interpreter(s). In the process of determining the number of image interpreters, which is performed prior to the process of assigning medical images to image interpreters, the controller 31 determines whether or not an abnormal shadow candidate is detected in the medical image. If no abnormal shadow candidate is detected in the medical image, a doctor who is in charge of the patient of the medical image is determined to be the image interpreter of the medical image.

Next, the controller 31 generates image interpreter assignment information in which the medical image is associated with the image interpreter(s) of the determined number.

If the controller 31 determines that the medical image cannot be associated with the image interpreter(s) of the number determined by the process of determining the number of image interpreters, the controller 31 may give a notice of this by, for example, outputting an alert.

### <Process of Outputting Image Interpreter Assignment Information>

Referring back to FIG. 5, the controller 31 outputs, to the image display apparatus 4, the image interpreter assignment information generated by the process of assigning medical images to image interpreters (Step S13).

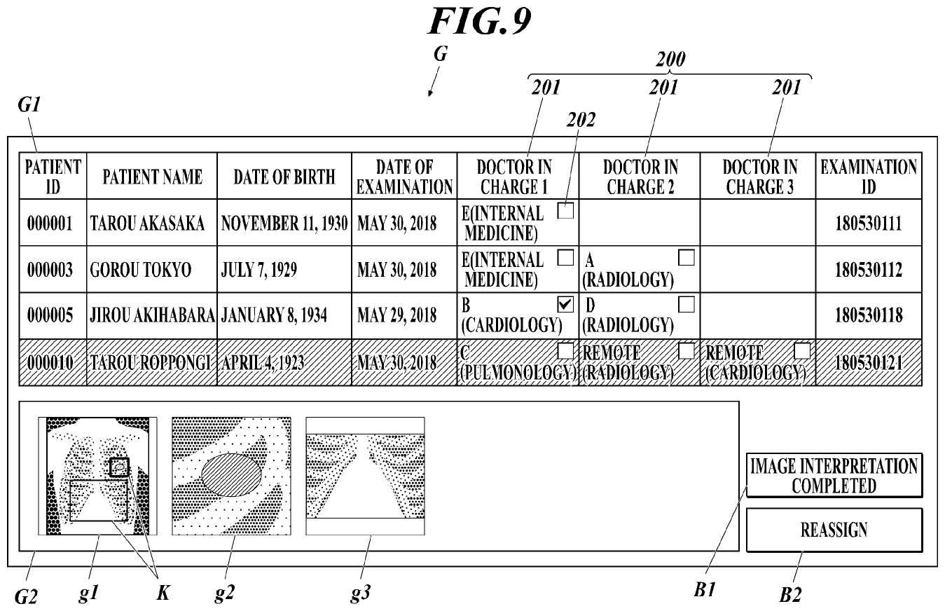

On the basis of the image interpreter assignment information, the image display apparatus 4 displays a list screen G (FIG. 9), and the image interpreter(s) interprets the medical image.

### <Process Thereinafter>

The controller 31 obtains a status of image interpretation of the medical image by the image interpreter(s) (Step S14), and determines whether or not a completion operation indicating that all image interpretation works by the image interpreters have been completed is performed (Step S15). If the controller 31 determines that the completion operation is performed (Step S15: YES), the controller 31 ends the series of processes.

More specifically, the controller 31 determines that the completion operation is performed if image interpretation reports by all the image interpreters determined to interpret the medical image have been registered. Alternatively, the controller 31 may determine that the completion operation is performed if any of the image interpreters operates an “image interpretation completed” button B1 (shown in FIG. 9) with the operation unit 42 of the image display apparatus 4.

The “image interpretation completed” button B1 is operated, for example, in a case where a plurality of image interpreters has been assigned the medical image, and any of the assigned image interpreters determines that the medical image does not need to be interpreted by a plurality of image interpreters. That is, the “image interpretation completed” button B1 is operated, for example, in a case where the CAD 2 has determined that a plurality of image interpreters should interpret the medical image, but any of the assigned image interpreters determines, in actual image interpretation, that the medical image does not need to be interpreted by a plurality of image interpreters.

If the controller 31 determines that the completion operation is not performed (Step S15: NO), the controller 31 determines whether or not an instruction operation to make an instruction to reassign an image interpreter(s) is input (Step S16). If the controller 31 determines that the instruction operation is input (Step S16: YES), the controller 31 returns to Step S11 and repeats the steps therefrom.

More specifically, the controller 31 determines that the instruction operation to make an instruction to reassign an image interpreter(s) is input if any of the image interpreters who has interpreted the medical image has operated a “reassign” button B2 (shown in FIG. 9) with the operation unit 42 of the image display apparatus 4.

The “reassign” button B2 is operated, for example, in a case where an image interpreter who has actually interpreted the medical image determines that the medical image should be interpreted by an image interpreter(s) in addition to the determined image interpreter(s). That is, the “reassign” button B2 is operated, for example, in a case where any of the assigned image interpreters determines, in actual image interpretation, that more image interpreters are required than the number determined by the information processing apparatus 3.

By specifying reassignment criteria (e.g. the number, the specialty, and the skill level of image interpreters to be added) in operating the “reassign” button B2, the process of determining the number of image interpreters (step S11) is performed in accordance with the specified criteria.

If the controller 31 determines that the instruction operation to make an instruction to reassign an image interpreter(s) is not input (Step S16: NO), the controller 31 lets the assigned image interpreter(s) continue interpreting the medical image, and repeats Step S15 and the processes thereafter.

Thus, management of the image interpreters is performed.

### [Process by the Image Display Apparatus 4]

The image display apparatus 4 performs a process of displaying medical images.

FIG. 8 shows a flowchart of the process of displaying medical images.

The controller 41 performs the process of displaying medical images in cooperation with the programs stored in the storage 45.

First of all, the controller 41 displays the list screen G on the display 43 in accordance with operations input by the operation unit 42 (Step S21).

More specifically, when an image interpreter logs in to the image display apparatus 4 with his/her ID, the log-in information is sent to the image DB 5 by the communication unit 44. In the image DB 5, the image management table is searched for the management information of the medical image for the log-in information (information on the image interpreter who logs in) on the basis of the image interpreter assignment information. Data of a list of medical images that match search criteria is generated and sent to the image display apparatus 4.

When receiving the data of the list with the communication unit 44, the image display apparatus 4 displays, on the display 43, the list screen G on the basis of the data of the list.

FIG. 9 shows an example of the list screen G.

As shown in FIG. 9, the list screen G has a list display part G1 and a thumbnail image display part G2.

The list display section G1 includes items of examination ID, patient ID, patient name, date of birth, date of examination, and doctor in charge 200. Cases are displayed on an examination basis. Although all the items are displayed in one row in the example here, the items may be displayed in a couple of rows.

The doctor in charge 200 includes a plurality of (here, three) doctor-in-charge display sections 201.

Each doctor-in-charge section 201 displays a name of a doctor (image interpreter) in charge and his/her department. The doctor-in-charge display sections 201 are filled as many as the number of the image interpreters determined to be in charge as the result of the process of assigning medical images to image interpreters. Only the department may be displayed here.

For example, with respect to a case in which an abnormal shadow candidate is not detected by the CAD 2, the name and the department of a doctor being in charge of the patient are displayed. The names and the departments of the image interpreters are displayed in order from an image interpreter who is in charge of an abnormal shadow candidate highest in the priority order.

Each doctor-in-charge display section 201 has a checkbox 202. When an image interpreter being displayed in the doctor-in-charge display section 201 completes interpreting the medical image, the image interpreter can check his/her checkbox 202. In a case where a detection result by the CAD 2 is determined to be highly accurate, the checkbox 202 may be automatically checked (interpretation of the medical image is determined to be complete) by the setting.

In the list display part G1, if all the image interpreters being displayed in the doctor in charge 200 have completed interpreting the medical image of a case, namely all the checkboxes 202 of the doctor-in-charge display sections 201 displaying the doctors in charge are checked, the case is deleted, or the display color of the row of the case is changed.

The doctor in charge may be changed by manipulation of the list display part G1. For example, it is possible to display a screen for selecting an image interpreter (not illustrated) when the department in the doctor-in-charge display section 201 is selected, and to change the doctor in charge when an image interpreter on the screen is selected.

Furthermore, when a case (patient ID or patient name) is selected in the list display part G1, a thumbnail image g1 and lesion-detected region images g2, g3 of the medical image can be displayed on the thumbnail image display part G2.

In the example in FIG. 9, the patient ID “000010” is selected in the list display part G1, and the thumbnail image g1 of the medical image of the case is displayed in the thumbnail image display part G2.

In the thumbnail image g1, abnormal shadow candidate regions are enclosed by frames K. The display colors of the frames K can be different according to the types of abnormal shadow candidates. The regions enclosed by the frames K in the thumbnail image g1 are displayed in an enlarged manner as the lesion-detected region images g2, g3.

The thumbnail image display part G2 allows a user to learn the detection result of abnormal shadow candidates and the reason why the medical image has been assigned to him/her before starting a screen viewer.

The list screen G also displays the “image interpretation completed” button B1 and the “reassign” button B2.

The “image interpretation completed” button B1 is operated, for example, in a case where a plurality of image interpreters has been assigned the medical image, and any of the assigned image interpreters determines that the medical image does not need to be interpreted by a plurality of image interpreters.

The “reassign” button B2 is operated, for example, in a case where any one of the assigned image interpreter(s) determines that the medical image should be interpreted by an image interpreter(s) in addition to the assigned image interpreter(s). There may be displayed an input section (not illustrated), if the “reassign” button B2 is operated, for specifying reassignment criteria (e.g. the number, the specialty, the skill level, etc. of an image interpreter(s) to be added).

If the “image interpretation completed” button B1 is operated, image interpretation completion information is included in image interpretation result information. If the “reassign” button B2 is operated, reassignment instruction information and reassignment criteria are included in the image interpretation result information.

Next, when a medical image to be interpreted is selected in the list screen G, the controller 41 sends a request, with the communication unit 44, to obtain the selected to-be-interpreted medical image to the image DB 5, and obtains the to-be-interpreted medical image and the CAD information of the medical image from the image DB 5 (Step S22).

In the image DB 5, when the request for the to-be-interpreted medical image is received, the to-be-interpreted medical image with the CAD information attached thereto is retrieved and sent to the image display apparatus 4.

When obtaining the to-be-interpreted medical image, the controller 41 displays, on the display 43, a viewer screen (not illustrated) that displays the obtained medical image (Step S23).

The viewer screen displays the to-be-interpreted medical image. To display the CAD information, by clicking a CAD information button or the like (not illustrated) for making an instruction to display an abnormal shadow candidate region(s) detected by the CAD 2, the CAD information is displayed over the medical image.

Next, when image interpretation result information is input by the image interpreter manipulating the operation unit 42, the controller 41 stores the image interpretation result information in the storage 45 (Step S24).

The input of the image interpretation result information is performed as follows as an example.

When the image interpreter specifies, with the operation unit 42, a lesion (focus) region that the image interpreter has determined to be a suspected lesion in the medical image displayed on the viewer screen, the viewer screen displays a mark indicating the lesion region over the medical image.

The viewer screen also displays an input section for the image interpreter to input findings on the specified lesion region. The input section has checkboxes for selecting a lesion type of the specified lesion region. When the image interpreter checks the lesion type with the operation unit 42, checkboxes for selecting findings (characteristics (e.g. small round, amorphous or indistinct, pleomorphic), categories, etc.) on the lesion region for the checked lesion type are displayed.

The input image interpretation result information is combined in accordance with lesion types, and image interpretation result information for each lesion type is generated. The image interpretation result information includes information on the lesion type, the number of lesion regions determined to be the lesion, positional information of each lesion region, and findings.

After the image interpretation result information has been input, the controller 41 displays the list screen G on the display 43 again, and sends the image interpretation result information to the information processing apparatus 3 through the communication unit 44 (Step S25).

Here, with respect to the image interpreter who has inputted the image interpretation result information, the checkbox 202 in his/her doctor-in-charge display section 201 is checked. One of the assigned image interpreters may operate the “image interpretation completed” button B1 or the “reassign” button B2.

The image interpretation result information includes, as described above, information on the lesion type, the number of lesion regions determined to be the lesion, positional information of each lesion region, and findings. The image interpretation result information may also include the image interpretation completion information, the reassignment instruction information and reassignment criteria.

The received image interpretation result information is associated with the medical image and stored in the image DB 5.

The image interpreter logs out of the image display apparatus 4 by manipulating the operation unit 42, and the controller 41 ends the process of displaying medical images. If interpretation of a medical image having a high triage level is left unattended when the image interpreter logs out, an alert may be output.

In this embodiment, when the image interpreter logs in to the image display apparatus 4 with his/her ID, the image management table is searched for the management information of the medical image for the log-in information (information on the image interpreter who logs in) on the basis of the image interpreter assignment information, and the data of the list of medical images that match search criteria is displayed on the display 43. However, a whole list of unattended cases may be displayed for the search as desired. The list of unattended cases is always updated, and if a case having high triage level comes in suddenly, the case can be displayed at the top of the list. Such an urgent case may be assigned to the image interpreter who is currently interpreting a medical image so that he/she can interpret the urgent case swiftly.

As described above, according to this embodiment, the controller 31 of the information processing apparatus 3: obtains an abnormal shadow candidate detection result (CAD information) generated based on a medical image obtained by the medical image generation apparatus 1; based on the obtained CAD information, determines the number of image interpreters who interpret the medical image; and outputs the determined number of image interpreters.

Because the number of image interpreters is determined for each medical image, in medical mage interpretation using the CAD, accuracy in image interpretation is improved without the overall working efficiency being decreased.

Furthermore, according to this embodiment, the CAD information includes information on whether or not an abnormal shadow candidate is present. In response to the abnormal shadow candidate being present according to the CAD information, the controller 31 determines the number of image interpreters to be one or plural. In response to the abnormal shadow candidate being absent according to the CAD information, the controller 31 determines the number of image interpreters to be one.

Thus, if an abnormal shadow candidate is present, the number of image interpreters is determined to be plural, and accuracy in image interpretation is improved.

Furthermore, according to this embodiment, the controller 31 generates image interpreter assignment information in which the medical image is associated with image interpreters of the determined number.

The controller 31 outputs the image interpreter assignment information to the image display apparatus 4. On the basis of the image interpreter assignment information, the image display apparatus 4 can display the list screen G.

Furthermore, according to this embodiment, the controller 41 of the image display apparatus 4 causes the display 43 to display the list screen G in which the medical image is associated with one or plural image interpreters on the basis of the image interpreter assignment information. Thus, with the list screen G, a user of the image display apparatus 4 can grasp at a glance the medical image and the image interpreters associated with the medical image.

Furthermore, according to this embodiment, the image interpreter assignment information includes information on the number of image interpreters assigned to interpret the medical image, the department in charge, and each of the assigned image interpreters; and the controller 41 causes the display 43 to display the list screen G in which the medical image is associated with the information on the number of image interpreters assigned to interpret the medical image, the department in charge, and each of the assigned image interpreters.

Thus, with the list screen G, the user of the image display apparatus 4 can grasp at a glance the medical image and the information on the number of image interpreters assigned the medical image, the department in charge, and each of the assigned image interpreters.

Furthermore, according to this embodiment, the image interpreters include an image interpreter who belongs to a medical facility different from a medical facility where the medical image has been taken. This makes it possible to interpret the medical image outside the medical facility where the medical image has been taken, and improves efficiency in image interpretation.

Furthermore, according to this embodiment, the controller 41 displays, in response to one of the cases in the list screen G being selected, an enlarged view of an abnormal shadow candidate region in the medical image of the selected case.

Thus, with the list screen G, the user of the image display apparatus 4 can roughly grasp the abnormal shadow candidate.

### Others

There are various modifications of the above embodiment. Hereinafter, the modifications are described mainly on points in which each modification differs from the above embodiment.

### First Modification

As a first modification, another mode of the process of determining the number of image interpreters is described.

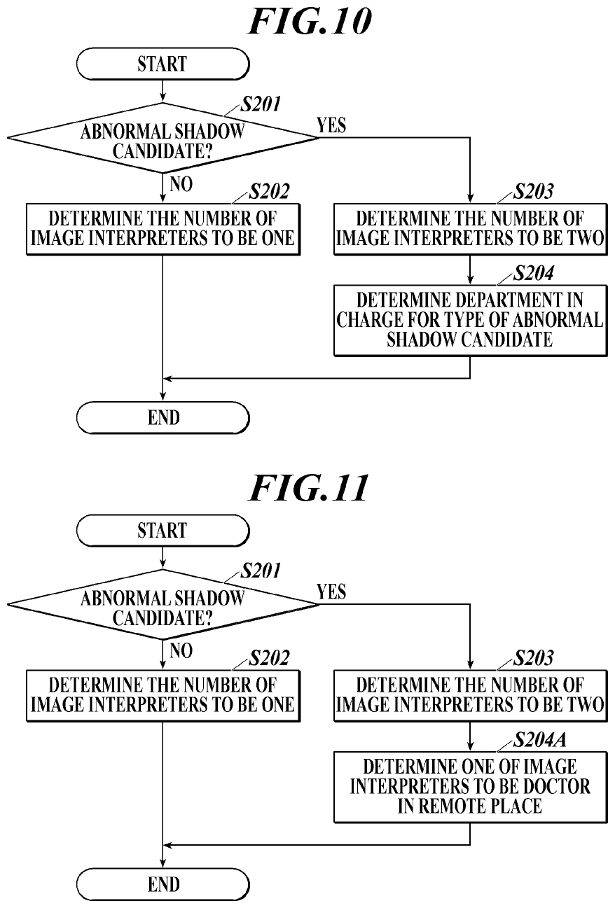

FIG. 10 shows a flowchart of the process of determining the number of image interpreters according to the first modification.

As shown in FIG. 10, the controller 31 determines whether or not an abnormal shadow candidate is present according to the CAD information (Step S201). If an abnormal shadow candidate is absent (Step S201: NO), the controller 31 determines the number of image interpreters to be one (Step S202). If an abnormal shadow candidate is present (Step S201: YES), the controller 31 determines the number of image interpreters to be two (Step S203), and determines a department in charge according to the type of the abnormal shadow candidate (Step S204).

In the process of determining the number of image interpreters, types of abnormal shadow candidates are associated with departments beforehand and stored in the storage 35. On the basis of the association information (see FIG. 7A), the department in charge is determined.

For example, if the type of abnormal shadow candidate is cardiomegaly, the department in charge is determined to be the department of cardiology, and if the type of abnormal shadow candidate is atelectasis, the department in charge is determined to be the department of pulmonology. If the medical facility does not have an appropriate department, the department in charge can be determined to be the appropriate department of a remote medical facility.

As shown in FIG. 11, if the controller 31 determines the number of image interpreters to be two (Step S203), one of the two image interpreters may be a doctor in a remote place (Step S204A).

In determining the department in charge for the abnormal shadow candidate, if the medical facility where the medical image has been taken does not have an appropriate department, an alert may be output.

According to the first modification, if an abnormal shadow candidate is present, interpretation of the medical image can be assigned to an appropriate department according to the type of the abnormal shadow candidate. Thus, the medical image can be accurately interpreted by a doctor with specialty in the field, and diagnostic accuracy is enhanced.

### Second Modification

As a second modification, another mode of the process of determining the number of image interpreters is described.

In the second modification, if the controller 31 determines that an abnormal shadow candidate is present but a certain criterion is met, the controller 31 determines the number of image interpreters to be one, not plural. For example, in many cases, a medical image(s) is taken in a normal examination on the basis of a request from a department being in charge of the patient. If the detected abnormal shadow candidate is in the domain of the department having made the request, the number of image interpreters can be determined to be one.

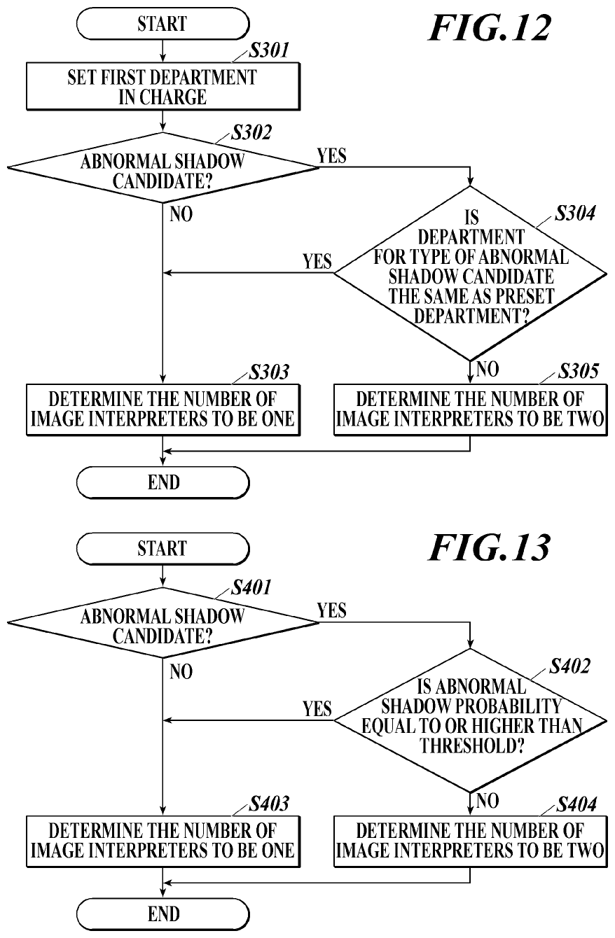

FIG. 12 shows a flowchart of the process of determining the number of image interpreters according to the second modification.

As shown in FIG. 12, the controller 31 sets the department that has made an imaging order to be a first department in charge (Step S301). The controller 31 then determines whether or not an abnormal shadow candidate is present according to the CAD information (Step S302). If an abnormal shadow candidate is absent (Step S302: NO), the controller 31 determines the number of image interpreters to be one (Step S303).

If an abnormal shadow candidate is present (Step S302: YES), the controller 31 selects the department in charge for the type of the abnormal shadow candidate on the basis of the information stored in the storage 35 and in which types of abnormal shadow candidates are associated with departments in charge (see FIG. 7A). The controller 31 then determines whether or not the department in charge is the same as a preset department, for example, the department having made the imaging order (the first department in charge set in Step S301) (Step S304). If the department in charge is the same as the preset department (Step S304: YES), the controller 31 determines the number of image interpreters to be one (Step S303).

If the department in charge for the type of the abnormal shadow candidate is different from the preset department (Step S304: NO), the controller 31 determines the number of image interpreters to be two (Step S305). The number of image interpreters may not be two but three or more, as desired.

In the flowchart in FIG. 12, it is possible to determine whether or not the department in charge for the type of the abnormal shadow candidate is the same as a preset department after determining the number of image interpreters to be two, and change the number of image interpreters from two to one if the department in charge for the type of the abnormal shadow candidate is the same as the preset department.

According to the second modification, increase of the number of image interpretation works is restrained, and diagnostic accuracy is secured.

### Third Modification

As a third modification, another mode of the process of determining the number of image interpreters is described.

FIG. 13 shows a flowchart of the process of determining the number of image interpreters according to the third modification.

As shown in FIG. 13, the controller 31 determines whether or not an abnormal shadow candidate is present according to the CAD information (Step S401). If the controller 31 determines that an abnormal shadow candidate is present (Step S401: YES), the controller 31 extracts the abnormal shadow probability from the obtained CAD information, and determines whether or not the abnormal shadow probability is equal to or higher than a predetermined threshold (Step S402).

If the abnormal shadow probability is equal to or higher than the predetermined threshold (Step S402: YES), the controller 31 determines the number of image interpreters to be one (Step S403). If the abnormal shadow probability is lower than the predetermined threshold (Step S402: NO), the controller 31 determines the number of image interpreters to be two (Step S404). Here, the number of image interpreters may not be two but three or more, as desired.

When detecting an abnormal shadow candidate, the CAD 2 also outputs certainty of determination on the abnormal shadow candidate. It is highly possible that a person also determines, with his/her eyes, that an abnormal shadow candidate having a high abnormal shadow probability is an abnormal shadow. Because such an obvious abnormal shadow candidate is hardly misinterpreted by an image interpreter interpreting the medical image alone, a plurality of image interpreters is not required for interpretation. In the third modification, if the abnormal shadow probability is equal to or higher than a predetermined threshold, the controller 31 determines the number of image interpreters to be one.

If the abnormal shadow probability is low, an image interpreter might somehow overlook the abnormal shadow candidate to be an abnormal shadow or misinterpret the abnormal shadow candidate. It is hence preferable that a plurality of image interpreters interpret the medical image. In the third modification, the number of image interpreters is increased for the medical image in which an abnormal shadow candidate having a low abnormal shadow probability has been detected.

According to the third modification, because the number of image interpreters is increased for the medical image that may be misinterpreted, accuracy in image interpretation is improved.

The display colors of rows in the list screen G may be changed according to the abnormal shadow probability so that a case of high importance can be visually recognized.

### Forth Modification

As a forth modification, another mode of the process of determining the number of image interpreters is described.

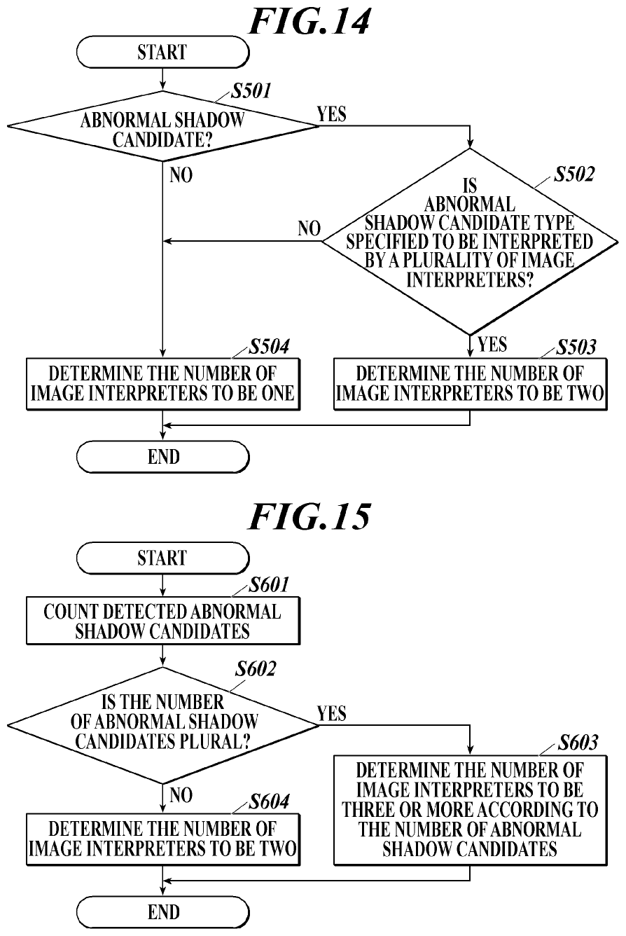

FIG. 14 shows a flowchart of the process of determining the number of image interpreters according to the forth modification.

As shown in FIG. 14, the controller 31 determines whether or not an abnormal shadow candidate is present according to the CAD information (Step S501). If an abnormal shadow candidate is present (Step S501: YES), the controller 31 determines whether or not the type of the abnormal shadow candidate is specified to be interpreted by a plurality of image interpreters in accordance with the number of image interpreters for each type of abnormal shadow candidate stored beforehand in the storage 35 (Step S502). If the type of the abnormal shadow candidate is specified to be interpreted by a plurality of image interpreters (Step S502: YES), the controller 31 determines the number of image interpreters to be two (Step S503). The number of image interpreters may not be two but three or more, as desired.

If the type of the abnormal shadow candidate is not specified to be interpreted by a plurality of image interpreters (Step S502: NO), the controller 31 determines the number of image interpreters to be one (Step S504).

That is, if the type of the abnormal shadow candidate is categorized as being high-risk in a case of being misinterpreted, such as a malignant tumor, a plurality of image interpreters interprets the medical image including such a high-risk abnormal shadow candidate. If the type of the abnormal shadow candidate is categorized as being low-risk, such as cardiomegaly, one image interpreter interprets the medical image including such a low-risk abnormal shadow candidate.

According to the forth modification, because the number of image interpreters is determined on the basis of the type of abnormal shadow candidate, accuracy in image interpretation is improved.

### Fifth Modification

As a fifth modification, another mode of the process of determining the number of image interpreters is described.

FIG. 15 shows a flowchart of the process of determining the number of image interpreters according to the fifth modification.

As shown in FIG. 15, the controller 31 counts abnormal shadow candidates in the medical image according to the CAD information (Step S601), and determines whether or not the number of abnormal shadow candidates is plural (Step S602). If the number of abnormal shadow candidates is plural (Step S602: YES), the controller 31 determines the number of image interpreters to be three or more according to the number of abnormal shadow candidates (Step S603).

If the number of abnormal shadow candidates is one (Step S602: NO), the controller 31 determines the number of image interpreters to be two (Step S604).

As described above, if the number of abnormal shadow candidates is one, two image interpreters interpret the medical image, and if the number of abnormal shadow candidates is two or more, three or more image interpreters interpret the medical image. This can decrease a risk that an abnormal shadow is unnoticed.

Here, if the number of abnormal shadow candidates is one, the number of image interpreters is determined to be two, and if the number of abnormal shadow candidates is two or more, the number of image interpreters is determined to be three or more. However, the number of image interpreters for each case can be set as desired.

In Step S602 of the fifth modification, the controller 31 may determine the number of image interpreters on the basis of the number of types of abnormal shadow candidates instead of the number of abnormal shadow candidates.

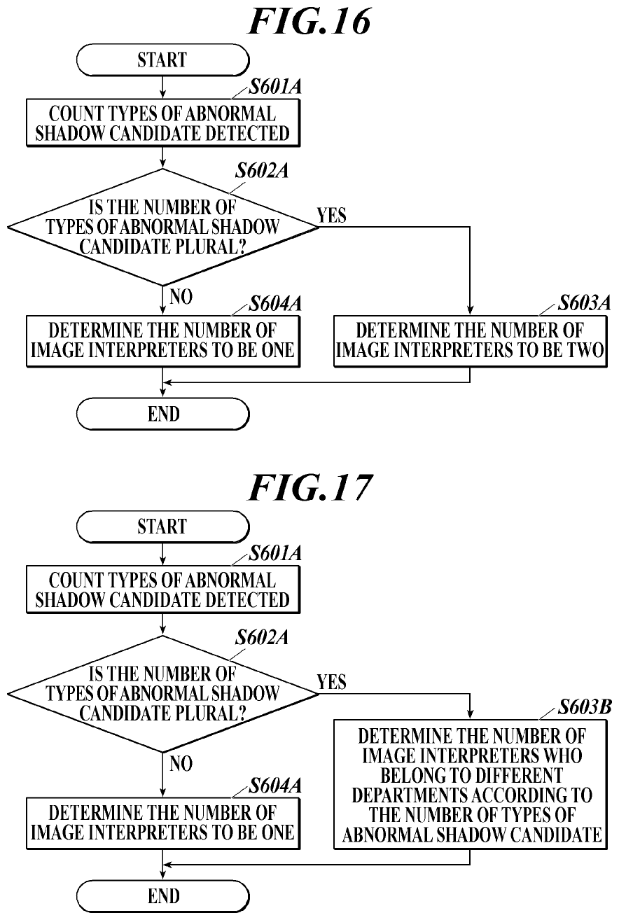

That is, as shown in FIG. 16, the controller 31 counts types of abnormal shadow candidates in the image (Step S601A) and determines whether or not the number of types of abnormal shadow candidates is plural (Step S602A). If the number of types of abnormal shadow candidates is plural (Step S602A: YES), the controller 31 determines the number of image interpreters to be two (Step S603A). If the controller 31 determines that the number of types of abnormal shadow candidates is one (Step S602A: NO), the controller 31 determines the number of image interpreters to be one (Step S604A).

As shown in FIG. 17, it is possible for the controller 31 to determine whether or not the number of types of abnormal shadow candidates is plural (Step S602A), and if the number of types of abnormal shadow candidates is plural (Step S602A: YES), determine the number of image interpreters who belong to different departments according to the number of types of abnormal shadow candidates (Step S603B).

As shown above, the number of image interpreters can be determined according to the number of types of abnormal shadow candidate, and the department in charge can be determined according to the type of abnormal shadow candidate. The maximum number can be set for the number of image interpreters because it is inefficient that unnecessarily many image interpreters interpret one medical image.

According to the fifth modification, the number of image interpreters is determined according to the number of abnormal shadow candidates or the number of types of abnormal shadow candidates. This can improve efficiency in image interpretation.

### Sixth Modification

As a sixth modification, another mode of the list screen G is described.

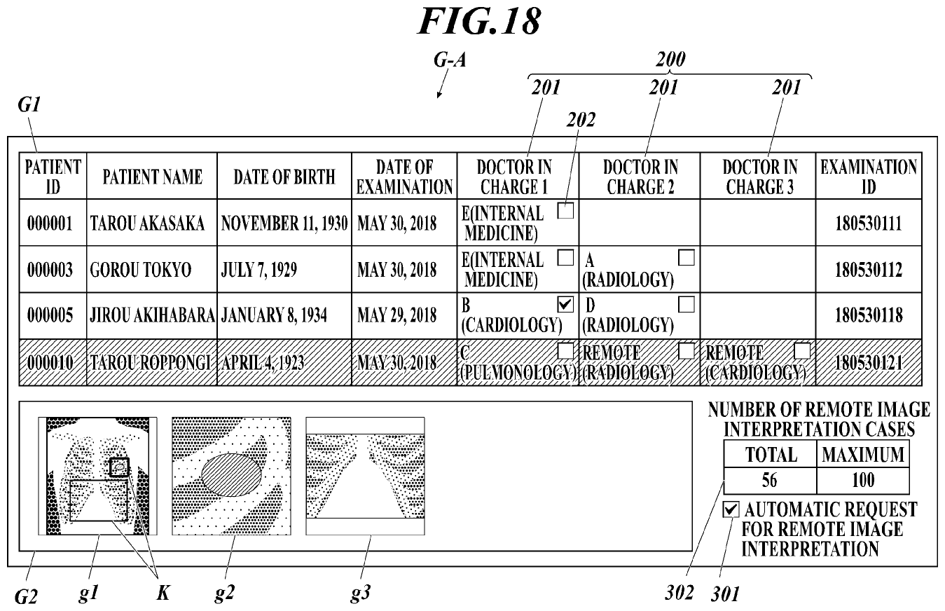

FIG. 18 is a list screen G-A according to the sixth modification.

As described above, the information processing apparatus 3 determines the number of image interpreters for a medical image, generates the image interpreter assignment information in which the medical image is associated with image interpreters of the determined number, and outputs the image interpreter assignment information to the image display apparatus 4. The image display apparatus 4 displays a list (list screen G) in which the medical image is associated with the image interpreters on the basis of the image interpreter assignment information.

The list screen G-A according to the sixth modification has a checkbox 301 for automatically requesting remote image interpretation. By checking the checkbox 301, remote image interpretation can be automatically requested for a medical image(s) in which an abnormal shadow candidate(s) has been detected by the CAD 2.

Furthermore, the list screen G-A has a display part 302 for displaying the maximum number and the total number of remote interpretation cases. If the maximum number of remote interpretation cases is determined, the maximum number is displayed in the display part 302. The automatic request for remote image interpretation of medical images, in which an abnormal shadow candidate(s) has been detected by the CAD 2, may not be accepted when the number of remote interpretation cases reaches the maximum.

According to the sixth modification, remote image interpretation can be automatically requested of image interpreters who belong to medical facilities different from the medical facility where the medical image has been taken.

This allows a plurality of medical facilities to cooperate with each other for making diagnoses, and consequently improves efficiency in image interpretation, and makes it possible to interpret more types of abnormal shadow candidates.

The embodiment and modifications first to sixth described above are some of preferred examples of the present invention and not intended to limit the present invention.

For example, although a chest X-ray image is cited as an example of the medical image in the above description, medical images of other body parts taken by other modalities may be used in the present invention.

Furthermore, in the above description, although an HDD and a nonvolatile memory are disclosed as examples of a computer-readable storage medium storing the programs for performing various processes, the computer-readable storage medium is not limited to these. As the computer-readable storage medium, a portable storage medium, such as a CD-ROM, may also be used. Also, as a medium that provides data of the programs via a communication line, a carrier wave can be used.

The detailed configurations and detailed operations of the apparatuses/components constituting the medical image display system 100 can be appropriately modified without departing from the scope of the present invention.

Although some embodiments of the present invention have been described and illustrated in detail, the disclosed embodiments are made for purposes of illustration and example only and not limitation. The scope of the present invention should be interpreted by terms of the appended claims.

The entire disclosure of Japanese Patent Application No. 2018-190165, filed on Oct. 5, 2018 is incorporated herein by reference in its entirety.

What is claimed is:

1. An information processing apparatus comprising a hardware processor that:
obtains an abnormal shadow candidate detection result generated based on a medical image obtained by a medical image generation apparatus;
based on the obtained abnormal shadow candidate detection result, determines the number of image interpreters who interpret the medical image; and
outputs the determined number of image interpreters.

2. The information processing apparatus according to claim 1, wherein
the abnormal shadow candidate detection result includes information on whether or not an abnormal shadow candidate is present,
in response to the abnormal shadow candidate being present according to the abnormal shadow candidate detection result, the hardware processor determines the number of image interpreters to be one or plural, and
in response to the abnormal shadow candidate being absent according to the abnormal shadow candidate detection result, the hardware processor determines the number of image interpreters to be one.

3. The information processing apparatus according to claim 2, wherein
the abnormal shadow candidate detection result includes information on a type of the abnormal shadow candidate, and
in response to the abnormal shadow candidate being present according to the abnormal shadow candidate detection result, the hardware processor determines the number of image interpreters to be plural, and determines a department in charge of interpreting the medical image according to the type of the abnormal shadow candidate.

4. The information processing apparatus according to claim 2, wherein
the abnormal shadow candidate detection result includes information on a type of the abnormal shadow candidate, and
the hardware processor determines, in response to the abnormal shadow candidate being present according to the abnormal shadow candidate detection result, the number of image interpreters to be:
one in response to a department in charge of interpreting the medical image that is determined according to the type of the abnormal shadow candidate being identical with a predetermined department; and
plural in response to the department in charge being different from the predetermined department.

5. The information processing apparatus according to claim 2, wherein
the abnormal shadow candidate detection result includes information on an abnormal shadow probability indicating a probability that the abnormal shadow candidate is an abnormal shadow, and
the hardware processor determines, in response to the abnormal shadow candidate being present according to the abnormal shadow candidate detection result, the number of image interpreters to be:
one in response to the abnormal shadow probability being equal to or higher than a predetermined threshold; and
plural in response to the abnormal shadow probability being lower than the predetermined threshold.

6. The information processing apparatus according to claim 2, wherein
the abnormal shadow candidate detection result includes information on a type of the abnormal shadow candidate, and
in response to the abnormal shadow candidate being present according to the abnormal shadow candidate detection result, the hardware processor determines the number of image interpreters according to the type of the abnormal shadow candidate.

7. The information processing apparatus according to claim 2, wherein
the abnormal shadow candidate detection result includes information on the number of abnormal shadow candidates about the abnormal shadow candidate, and
in response to the abnormal shadow candidate being present according to the abnormal shadow candidate detection result, the hardware processor determines the number of image interpreters to be two or more according to the number of abnormal shadow candidates about the abnormal shadow candidate.

8. The information processing apparatus according to claim 2, wherein
the abnormal shadow candidate detection result includes information on the number of types of the abnormal shadow candidate, and
in response to the abnormal shadow candidate being present according to the abnormal shadow candidate detection result, the hardware processor determines the number of image interpreters according to the number of types of the abnormal shadow candidate.

9. The information processing apparatus according to claim 8, wherein the hardware processor determines a department in charge of interpreting the medical image according to the number of types of the abnormal shadow candidate.

10. The information processing apparatus according to claim 1, wherein the hardware processor:
generates image interpreter assignment information in which the medical image is associated with one or more image interpreters of the determined number; and
outputs the image interpreter assignment information.

11. The information processing apparatus according to claim 10, wherein the hardware processor gives a notice in response to determining that the medical image cannot be associated with the one or more image interpreters of the determined number.

12. A medical image display apparatus that connects to the information processing apparatus according to claim 10 and the medical image generation apparatus and displays the medical image, the medical image display apparatus comprising a hardware processor that causes a display to display a list screen of a case in which the medical image is associated with one or more image interpreters based on the image interpreter assignment information.

13. The medical image display apparatus according to claim 12, wherein
the image interpreter assignment information includes information on the number of image interpreters assigned to interpret the medical image, a department in charge of interpreting the medical image, and each of the one or more image interpreters assigned; and
the hardware processor causes the display to display the list screen of the case in which the medical image is associated with the information on the number of image interpreters assigned to interpret the medical image, the department in charge, and each of the assigned image interpreters.

14. The medical image display apparatus according to claim 12, wherein the one or more image interpreters include an image interpreter who belongs to a medical facility different from a medical facility where the medical image has been taken.

15. The medical image display apparatus according to claim 12, wherein in response to a case being selected from the case in the list screen, the hardware processor displays an enlarged view of an abnormal shadow candidate region in the medical image of the selected case.

16. A non-transitory computer-readable storage medium storing a program to cause a computer to:
obtain an abnormal shadow candidate detection result generated based on a medical image obtained by a medical image generation apparatus;
based on the obtained abnormal shadow candidate detection result, determine the number of image interpreters who interpret the medical image; and
output the determined number of image interpreters.
